(19) United States
(12) Patent Application Publication
(10) Pub. No.: US 2011/0171710 A1
(43) Pub. Date: Jul. 14, 2011

(54) METHOD FOR PRODUCING CELLULOSIC ETHANOL

(75) Inventors: Xuefeng Yu, Hubei (CN); Zhihong Li, Hubei (CN); Minghua Yu, Hubei (CN); Juan Yao, Hubei (CN); Zhijun Li, Hubei (CN); Daiwu Liu, Hubei (CN)

(73) Assignee: ANGELYEAST CO., LTD, Yichang, Hubei (CN)

(21) Appl. No.: 13/054,851

(22) PCT Filed: Sep. 27, 2009
(86) PCT No.: PCT/CN2009/074249

§ 371 Date: Apr. 5, 2011

(30) Foreign Application Priority Data
Dec. 24, 2008 (CN) 200810189465.0

(51) Int. Cl. C12P 7/10

(52) U.S. Cl. 435/165

(57) ABSTRACT

A method for producing cellulosic ethanol is disclosed, which comprises the following steps: adding the medium containing the source materials of cellulose and/or hemicellulose to fermentation reaction vessel; adding cellulase to fermentation reaction vessel, and inoculating Candida lusitaniae; running simultaneous saccharification fermentation (SSFs) with the combined action of cellulose and Candida lusitaniae, and obtaining cellulosic ethanol by separation. Candida lusitaniae used in SSFs in present invention has higher ethanol tolerance and a higher ethanol production rate, it can use cellulose and hemicellulose as source materials and effectively produce cellulosic ethanol with cellulase in SSFs.

### FIELD

The invention involves the technical field of exploitation of bioenergy, and in particular involves a method for producing cellulosic ethanol.

### BACKGROUND

As petroleum resources exhaust, oil prices rise, and the environment deteriorates, looking for renewable resources to replace fossil resource and exploiting clean energy resources has been a focus in the industrial biotechnology field. The exploitation of biomass energy can diffuse the dependence on petroleum resources, control carbon dioxide emissions, and promote the development of new industry chains in agriculture, which plays an important role in promoting economic development.

Fuel ethanol, as a renewable clean resource, has been developing in America and Brazil since at least the 1970's. The productivity of ethanol has been reached more than 5,000,000 tons per year in our country, wherein the annual yield of fuel ethanol remains about 1,100,000 tons, only second to Brazil, America, ranking the third in the world.

A great deal of corn has been used as a resource to exploit bioenergy, which causes corn prices to increase continually, so, after the upsurge of turning foodstuff into ethanol, the exploitation of cellulosic ethanol techniques has become a direction for large-scale commercialization of cellulosic ethanol.

Lignocellulose material is one of the largest renewable resources on the earth, including forest, crop straw, the processing remains of agricultural by-products etc., which enters on the important place in the energy flows and material recycle flows of natural ecosystem. It is a potential resource to produce fuel ethanol, and thus a possible answer of energy demand in the future.

Currently, it has prominent issue for the techniques in pilot-scale of cellulosic ethanol. Firstly, the commercialized industrial yeast on the market can ferment glucose and maltose, but not cellulosic disaccharide. The expensive β-glucosidase, besides cellulase, needs to be added in the production process, in order to avoid the inhibition of cellulosic disaccharide. Cellulase is needed to hydrolyze cellulose into oligosaccharide. One of these oligosaccharides is cellulosic disaccharide, which includes two glucose molecules which can be further disassembled into glucose. However, cellulose does not have enough β-glucosidase to turn cellulosic disaccharide disassembled from the hydrolysis of cellulose into glucose. Cellulosic disaccharide can inhibit endoglucanase and exoglucanase, and reduce the production rate and yield of the whole ethanol in Simultaneous Saccharification and Fermentation (SSFs). Furthermore, a great deal of the expensive β-glucosidase needs to be supplemented, which results in remarkably high cost. On the other hand, the yeast to ferment cellulosic disaccharide has been found, but it has a very low ethanol tolerance.

Secondly, SSFs cannot guarantee that enzymolysis temperature harmonizes with the fermentation temperature. Generally, the enzymolysis temperature is 40-50° C., while the fermentation temperature of Saccharomyces cerevisiae is 28-35° C., thus it has to address the compromise in the process, resulting in excessive amounts of enzyme or yeast, generally with the time in SSFs more than 100 hours, lower fermentation efficiency. Thirdly, under the terms of high content of substrate, a large quantity of cellulose is still not converted, the effect of ezymolysis decreases remarkably, and the yield of ethanol is also too low, generally only around 3-5% (v/v).

The preparation rate and the yield of ethanol fermentation at present cannot economically meet the requirements of SSFs. A Chinese national patent (CN 101130792A) reported a method to prepare fuel ethanol by ezymolyzing straw, wherein fermentation time lasted 10-15 days and the efficiency of ezymolysis was remarkably low. Therefore, there is no feasibility of industrialization. Another Chinese national patent (CN101270372A) reported a method to concentrate sugar solution in fermentation liquid of cellulosic ethanol which aimed to resolve the problem of too low sugar concentration after enzymolysis. This shows the current enzymolysis technology cannot meet the requirements of industrialization. Although this patent technology involving concentration can be utilized to change the enzymolyzed sugar concentration, the problem of the low enzymolysis rate of cellulose is not resolved yet, which can cause problems of low utilization rate of raw materials and high production cost. Another Chinese national patent (CN 101230359) reported a method to concentrate cellulosic ethanol by a modified polydimethyl siloxane polymer membrane, which also wanted to resolve the problem of too low concentration of fermentation ethanol and high cost of distillation, but no way to avoid low efficiency of ezymolysis and fermentation.

### SUMMARY

The following simplified summary is provided in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

The aim of the present invention is to provide a method to produce cellulosic ethanol, by increasing the efficiency of SSFs, thus increasing the production rate and the yield of cellulosic ethanol.

To achieve the aim of the present invention, the technical solutions are supplied as follows:

A method for producing cellulosic ethanol, which comprises the following steps:

1) adding the medium containing the source materials of cellulose and/or hemicelluloses to fermentation reaction vessel;

2) adding cellulase to fermentation reaction vessel, and inoculating Candida lusitaniae;

3) conducting simultaneous saccharification fermentation (SSFs) with the combined action of cellulose and Candida lusitaniae, and obtaining cellulosic ethanol by separation.

Wherein, the origin of the source materials of cellulose and/or hemicelluloses in step 1) are renewable lignocellulose biomass. Such as wheat straw, rice straw and fast growing forest etc. are used as the source materials of cellulosic ethanol. This biomass contains 20-70% cellulose and 10-40% hemicelluloses of plants dry weight, and partial lignin. At the same time, it also includes all kinds of biomass which are rich in cellulose and discarded from the environments and living goods.

Wherein, the said medium in step 1) contains 15%-30% cellulose and/or hemicelluloses source materials, still contains a little yeast extracts, peptone, antibiotics. The pH value is controlled to 3.5-6.0. The medium can be added Tween 80 and ergosterol etc according to the properties of liquids.

Wherein, cellulase in step 2) contains endoglucanase, exoglucanase and a little β-glucosidase.

Wherein, Candida lusitaniae in step 2) prefers the yeast which is domesticated by ethanol and cellulosic disaccharide. The medium used for domestication contains cellulosic disaccharide and proper amounts of ethanol. 5%, 10%, 15% three concentration grades of cellulosic disaccharide are used for domestication, separated by underlines generation after generation, and then 1%, 2%, 3% ethanol grades are used for domestication, obtaining advantageous strains by separation.

Wherein, pH value in SSFs is 3.5-6.0, the reaction temperature is 35-45° C., preferred 40-45° C.

Candida lusitaniae used in SSFs in the present invention has higher ethanol tolerance and a higher ethanol production rate, which can use cellulose and hemicelluloses as source materials and effectively produce cellulosic ethanol with cellulase in SSFs. The tests show that Candida lusitaniae has a higher ethanol production rate and a higher yield by the comparison of the fermentation of other yeast using glucose and cellulosic disaccharide; in addition, compared with other yeasts, Candida lusitaniae has higher ethanol tolerance.

To the accomplishment of the foregoing and related ends, certain illustrative aspects are described herein in connection with the following description and the annexed drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

FIG. 1 shows a comparison of productivity of ethanol in SSFs between different kinds of yeast and Candida lusitaniae.

FIG. 2 shows a comparison of ethanol tolerance of different kinds of yeast.

### DETAILED DESCRIPTION

The present invention is detailed by the following modes of carrying out the invention.

The fermentation source materials involved in the present invention contain cellulose and/or hemicelluloses. The origin is renewable lignocellulose biomass, such as wheat straw, rice straw and fast growing forest etc., which are all used as the source materials of cellulosic ethanol. This biomass contains 20-70% cellulose and 10-40% hemicelluloses by plants dry weight, and partial lignin. At the same time, it also includes all kinds of biomass which are rich in cellulose and discarded from the environments and living goods.

Cellulase used in the present invention contains endoglucanase, exoglucanase and a little β-glucosidase. Cellulase is used to hydrolyze cellulose into oligosaccharides, such as glucose. One of these oligosaccharides is cellulosic disaccharide, including two molecules of glucose, which can dissemble into glucose. β-glucosidase can turn cellulosic disaccharide hydrolyzed from cellulose into glucose, but cellulase only contains a little β-glucosidase, thus cellulase itself cannot utilize cellulosic disaccharide sufficiently.

Candida lusitaniae used in the present invention can utilize cellulose hydrolysis products, cellulosic disaccharide and glucose, also can utilize mannose and galactose hydrolyzed from hemicelluloses, and the above combined sugar and other related sugar also can utilize it to ferment. Wherein, said disaccharides can be sucrose, maltose, lactose and cellulosic disaccharide, but not including melibiose and fucose. The monosaccharide hydrolyzed from polysaccharide can also be utilized, for example starch and glucose hydrolyzed from cellulose and other monosaccharides, such as fructose, sorbose, mannose and galactose.

The following illustrates concretely the steps and processes to effectively produce cellulosic ethanol in SSFs involved in the present invention.

1. The Cultivation and Domestication of Candida lusitaniae

Candida lusitaniae separated from soil samples enriches in potato agar medium. This yeast strain is purebred in biology, which is appraised by Candida lusitaniae (CLAS 5566). One sample is conserved in the conservation centre of Alan G. Mac Diarmid Research Institute of Renewable Energy, Three Gorges University. The medium used for domestication contains cellulosic disaccharide and proper amounts of ethanol. 5%, 10% and 15% three concentration grades are used for domestication, separated by underlines generation after generation, and then 1%, 2% and 3% ethanol grades are used for domestication, obtaining advantageous strains by separation.

2. Fermentation Conditions

Dissolved Oxygen Control of Fermentation Process

The required scope in the fermentation process is wide. Dissolved oxygen can be either micro-oxygenation in interrupted fermentation, or a little air ventilated through the substrate inoculated in continuous fermentation. Furthermore, it can also be anaerobic fermentation. The required technique will depend on the initial cell density, substrate concentration and inoculation conditions.

The Formula of Fermentation Medium

The medium used for fermentation is normal medium, which contains proper nitrogen source, minerals, vitamins and carbon sources. These carbon sources include hexoses (glucose, galactose and mannose) and disaccharides (cellulosic disaccharide). The medium used for fermentation and inoculation mainly includes 18% cellulose, 1% Angel yeast extract, 2% peptone and 2 mg/L antibiotics (pH value is 3.5-6.0). The medium can be added Tween 80 and ergosterol etc. according to the properties of liquids.

The Temperature Control of Fermentation Process

The temperature of this fermentation process can range from 28° C. to 45° C., however the optimum temperature is 40-45° C.

3. Simultaneous Saccharification Fermentation (SSFs)

In the process in SSFs, the saccharification involves the hydrolysis process of cellulose. Cellulase is utilized to hydrolyze cellulose into oligosaccharides. One of these oligosaccharides is cellulosic disaccharide, which includes two glucose molecules, and can be disassembled into glucose.

The yeast strain used in the present invention is Candida lusitaniae, which can ferment cellulosic disaccharides very well at 42° C., can be used in SSFs to provide faster production rate of wine and higher ethanol yield, superior to the known other yeasts used for the fermentation of cellulosic disaccharide.

By comparing the effect of SSFs on the final wine production with that of interrupted saccharification fermentations(ISFs), the results in Table 1 show that the utilization rate of cellulose in SSFs has been increased by 50% when compared with that in ISFs, and yeast efficiency has been increased twice.

TABLE 1
The effects of SSFs and ISFs on the final wine production

| | SSFs process | ISFs process |
|---|---|---|
| saccharification rate | 85.1% | 58.5% |
| residual reducing sugar (v/v) | 0.15% | 0.235% |
| alcohol degree (v/v, 20° C. ) | 10.2% | 5.3% |

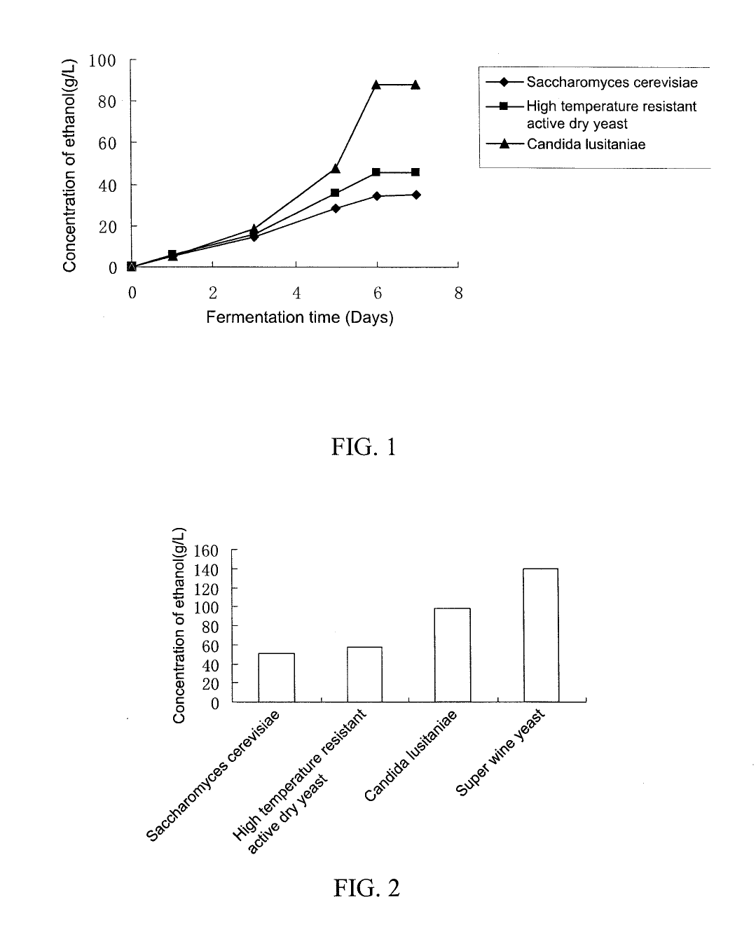

FIG. 1 illustrates the data in SSFs of different yeast strains. CLAS5566 is compared with other yeast strains, Saccharomyces cerevisiae, and high temperature resistant active dry yeast after 6 days later, the concentration of ethanol utilizing yeast strain CLAS 5566 attained about 90 g/L. It is clear to see that it is superior to other yeast strains in rates and yields by comparing with other yeast strains.

FIG. 2 shows that at 40° C., CLAS 5566 obtained from 25% cellulose has a ethanol resistant concentration attaining to 100 g/L. The ethanol tolerance is remarkably superior to that of Saccharomyces cerevisiae and high temperature resistant active dry yeast, a little lower than that of super wine yeast from Angel company.

### Example 1

Adding the medium containing cellulose to a 6 L fermentation reaction vessel filled with 2 L water, the sample amount reaches 2.5 L, to ensure the enough space while inoculating. The medium mixes in the fermenter containing lipids (30 ml/L oleic acid) and sterilizes for 30-40 minutes at 120° C. Antibiotics contains 500 mg penicillin with the concentration of 10 mg/L and 500 mg (10 mg/L) streptomcycin (pH value is 4.5-5.0). In succession, cellulase is added and CLAS 5566 special yeast for cellulosic ethanol is inoculated. Cell density is 2×10⁷, and the solution is diluted to 3 L with water. Enzymes destroy cellulose into glucose and cellulosic disaccharide, and the yeast ferments into ethanol. The temperature is controlled at 38° C., simultaneous saccharification ferments for 72 hours, to obtain 89 g/L ethanol, and then ethanol is fractionated from fermentation substrates, to obtain cellulosic ethanol.

Scale Preparation Example 2

Adding the medium containing cellulose to a 20 L fermentation reaction vessel filled with 4 L water, the sample amount reaches 5.5 L, to ensure the enough space while inoculating. The medium mixes in the fermenter, containing lipids (ergosterol 5 mg/L) sterilizes for 30-40 min at 120° C. Antibiotics contains 500 mg penicillin with the concentration of 10 mg/L and 500 mg (10 mg/L) streptomcycin (pH value is 4.5-5.0). In succession, cellulase is added, and CLAS 5566 special yeast for cellulosic ethanol is inoculated. Cell density is 2×10⁸, and the solution is diluted to 5 L with water. Enzymes destroy cellulose into glucose and cellulosic disaccharide, and the yeast ferments into ethanol. The temperature is controlled at 42° C. and simultaneous saccharification ferments for 96 hours, to obtain 98 g/L ethanol, and then ethanol is fractionated from fermentation substrates, to obtain cellulosic ethanol.

What has been described above includes examples of one or more embodiments. It is, of course, not possible to describe every conceivable combination of components or methodologies for purposes of describing the aforementioned embodiments, but one of ordinary skill in the art may recognize that many further combinations and permutations of various embodiments are possible. Accordingly, the described embodiments are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method for producing cellulosic ethanol, comprising:
adding a medium containing source materials of cellulose and/or hemicelluloses to a fermentation reaction vessel;
adding cellulase to the fermentation reaction vessel, and inoculating Candida lusitaniae; and
conducting simultaneous saccharification fermentation (SSFs) with the combined action of cellulose and Candida lusitaniae, and obtaining cellulosic ethanol by separation.

2. The method of claim 1, wherein, the origin of the source materials of cellulose and/or hemicelluloses is renewable lignocellulose biomass.

3. The method of claim 1, wherein the medium contains 15% to 30% cellulose and/or hemicelluloses source materials.

4. The method according of claim 3, wherein the medium contains at least some yeast extracts, peptone and antibiotics.

5. The method of claim 1, wherein the pH value of the medium is controlled within the range of 3.5-6.0.

6. The method of claim 1, wherein the cellulase contains endoglucanase, exoglucanase and at least some β-glucosidase.

7. The method of claim 1, wherein the Candida lusitaniae in is a yeast that is domesticated by ethanol and cellulosic disaccharide.

8. The method of claim 7, wherein, 5%, 10% and 15% concentration grades of cellulosic disaccharide are used for domestication, separated by underlines generation after generation, and then 1%, 2% and 3% ethanol grades are used for domestication, obtaining advantageous strains by separation.

9. The method according to claim 1, wherein, the reaction temperature of simultaneous saccharification fermentation (SSFs) is 35-45° C.

10. The method according to claim 9, wherein, the reaction temperature of SSFs is 40-45° C.
